(19) United States
(12) Patent Application Publication
(10) Pub. No.: US 2006/0153789 A1
(43) Pub. Date: Jul. 13, 2006

(54) Stimulation of hair regrowth

(76) Inventor: Mark L Witten, Tucson, AZ (US)

Correspondence Address: BANNER & WITCOFF, 1001 G STREET N W, SUITE 1100, WASHINGTON, DC, 20001, US

(21) Appl. No.: 10/539,734

(22) PCT Filed: Dec. 18, 2003
(86) PCT No.: PCT/US03/40259

§ 371 Date: Dec. 21, 2005

Related U.S. Application Data
(60) Provisional application No. 60/433,996, filed on Dec. 18, 2002.

(51) Int. Cl. A61K 8/00

(52) U.S. Cl. 424/70.1

(57) ABSTRACT

Hair loss is treated by administration of Substance P or a bioactive analog thereof. The subject may have loss of hair due to any of a variety of reasons, including chemotherapy, stress, radiation, male pattern baldness, nutritional deficit. The treatment can be conveniently administered by aerosol.

This application claims the benefit of provisional application Ser. No. 60/433,996 filed 18 Dec. 2002, the disclosure of which is expressly incorporated herein.

### TECHNICAL FIELD OF THE INVENTION

This invention is related to the area of hair growth. In particular, it relates to retarding and/or reversing hair loss.

### BACKGROUND OF THE INVENTION

Hair or pili are fine threadlike appendages of the skin which normally cover the entire body (with the exception of the palms of the hands and soles of the feet, and the flexor surfaces of joints). A hair comprises a root embedded in a hair follicle and a free portion (the stem or shaft). The term hair refers to both mature hair as well as the soft, downy hair known as vellus hair.

The hair bulb or follicle is a compact structure located in the dermis layer of the skin and is composed of three main cellular groups. The first comprises a compact group of fibroblasts known as the dermal papilla which includes a capillary system. The second group comprises germinative epithelial cells of the hair bulb which proliferate and differentiate to give rise to the mature hair shaft. The third group of fibroblasts exists around the outside of the bulb in the connective tissue sheath.

Hair loss occurs in many pathological conditions. Hair loss is commonly divided into two categories, cicatricial (scarring) and noncicatricial alopecia. Cicatricial alopecia results from hair follicle damage and pathological changes of the surrounding skin. Noncicatricial alopecia is caused by either functional or structural disorders of the hair follicle itself. Noncicatricial alopecia can result from chemotherapy or radiation treatments of cancers, nutritional and hormonal disorders, or stress. Male pattern alopecia (androgenic alopecia) and alopecia aceata are common hair loss afflictions.

Humans typically have 100,000 to 150,000 hairs, and it is normal to lose 50 to 150 hairs daily. The maintenance of the typical amount of hair depends on the pilar cycle, during which the hair forms, grows, and falls out before being replaced by a new hair which appears in the same follicle. During a pilar cycle, three phases are successively observed, namely, the anagen phase, the catagen phase and the telogen phase. During the anagen phase, the hair undergoes a period of active growth associated with intensive metabolic activity in the bulb. The catagen phase is transitory and is marked by a slowing-down of mitotic activity. During this phase, the hair undergoes involution and the follicle atrophies. The telogen phase corresponds to a period of rest of the follicle and shedding of the hair. The old hair is pushed by an incipient anagen hair. This process of continuous physical renewal undergoes a natural change during aging; the hairs become thinner and their cycles shorter.

Alopecia occurs when the pilar cycle is accelerated or disturbed. For example, when the growth phases are shortened, the hairs proceed to the telogen phase earlier and they are shed in larger numbers. The successive growth cycles lead to increasingly thinner and increasingly shorter hairs, converting gradually to an unpigmented down. This phenomenon may lead to baldness. The pilar cycle is dependent on many factors which are capable of causing more or less pronounced alopecia. Diet, endocrine function, nervous status, etc., may play a role.

The active anagenic phase or growth phase lasts several years during which the hair grows longer. It is followed by a very short and transitory catagenic phase, which lasts a few weeks. Finally the telegenic phase lasts a few months. At the end of the resting period, the hair falls out and another cycle begins. The head of hair is thus constantly renewed and, of the approximately 150,000 hairs which a head of hair contains, at any time approximately 10% of them are at rest and will therefore be replaced in a few months. In a significant number of cases, early hair loss takes place in subjects who are genetically predisposed to it and it affects men in particular. It is more particularly androgenic in character or is referred to as androgenic alopecia. This alopecia is essentially due to a disturbance in hair renewal which results in an acceleration in the frequency of the cycles at the expense of the quality of the hair and subsequently of its amount. A progressive thinning of the head of hair takes place by regression of the so-called “terminal” hairs to the downy stage. Regions are preferentially affected, in particular the temple or frontal bulbs in men and, in women, a difffuse alopecia of the vertex is observed. The term alopecia includes a host of disturbances of the hair follicle, whose final consequence is the partial or permanent loss of hair.

There is a continuing need in the art for substances which suppress or reduce the effect of alopecia, retarding hair loss and/or inducing new hair growth.

### SUMMARY OF THE INVENTION

According to the invention a method is provided for inducing hair growth or retarding hair loss. Substance P or a bioactive analog thereof is administered to a subject in need thereof. The bioactive analog is selected from the group consisting of [Met-OH¹¹]-substance P, [Met-OMe¹¹]-substance P, [Nle¹¹]-substance P, [Pro⁹]-substance P, [Sar⁹]-substance P, [Tyr⁸ ]-substance P, [p-Cl-Phe^{7,8}]-substance P, [Sar⁹,Met (0₂)¹¹]-substance P, and analogs having the amino acid backbone RPKPQQFFGLM-NH₂.

Hair growth is thereby induced or hair loss is retarded.

These and other embodiments which will be apparent to those of skill in the art upon reading the specification provide the art with reagents and methods for treating hair loss.

### BRIEF DESCRIPTION OF THE DRAWINGS

FIG. 1A. A C57BL/6 mouse irradiated with 7.5 Gy gamma radiation on Sep. 24, 2003 and given 12 days of [Sar, Met(0₂)¹¹]-substance P (Homspera™) treatment.

FIG. 1B. A C57BL/6 mouse irradiated with 7.5 Gy gamma radiation on Sep. 24, 2003 and given no Homspera™ treatment.

FIG. 2A. Group 1 (90 day treatment of Homspera™) mice photographed at day 56 after radiation.

FIG. 2B. Group 2 (35 day treatment of Homspera™) mice photographed at day 56 after radiation.

FIG. 2C. Group 1 (90 day treatment of Homspera™) mice photographed at day 90 after radiation.

FIG. 2D. Group 2 (35 day treatment of Homspera™) mice photographed at day 90 after radiation.

### DETAILED DESCRIPTION OF THE INVENTION

The inventors have discovered that Substance P (SP) stimulates hair regrowth and/or retards hair loss. This discovery can be applied to any of the many diverse causes of hair loss, including but not limited to androgenic alopecia and drug or radiation induced alopecia. Successful treatment results in an increase in the number of hairs, rather than in the length of hairs.

Aerosolization has been found to be a very effective means of administering Substance P to mammalian subjects. However, other means, as are known in the art, such as intravenous, subcutaneous, intramuscular, intraperitoneal, transdermal, topical, and intraarterial administration can be used as alternatives. Any such means as is known in the art can be applied.

Substance P (RPKPQQFFGLM-NH₂) or any of its bioactive analogues can be used in the methods of the present invention. These include, but are not limited to:. [Met-OH¹¹]-substance P, [Met-OMe¹¹]-substance P, [Nle¹¹]-substance P, [Pro⁹]-substance P, [Sar⁹]-substance P, [Tyr⁸]-substance P, [p-Cl-Phe^{7,8}]-substance P, and [Sar, Met(0₂)¹¹]-substance P, and other analogs which have the amino acid backbone RPKPQQFFGLM-NH₂. Bioactive analogs according to the invention are those which act as competitive inhibitors of SP by binding to the SP receptor (NK-1 receptor). Other derivatives as are known in the art and commercially available (e.g., from Sigma) can be used. In addition, substance P fragments and derivatized substance P fragments may also be used. Substitution, deletion, or insertion of one to eight amino acid residues, and preferably from one to three amino acid residues, will lead to analogs which can be routinely tested for biological activity. In addition, functional groups may be modified on SP while retaining the same peptide backbone. Again, routine testing will determine which of such modifications do not adversely affect biological activity.

Typical concentration ranges of substance P or its bioactive analogue in the aerosol administered is between 0.001 and 50 μM. Concentrations in the range of between 0.05 and 5 μM are particularly useful. It can be advantageously administered as a liquid at a concentration between about 0.1 and 10 μM.

The method of the present invention is useful in the treatment of alopecia in mammals, and as such may be used to promote, increase, or assist in the growth of hair. Subjects may be male or female. The term alopecia refers to both the complete absence of hair in skin which typically exhibits hair growth, as well as to a loss or diminution in the amount of hair. Multiple types and causes of alopecia are recognized in humans, including male pattern baldness, chemotherapy induced hair loss, congenital alopecia, and alopecia areata. The term treating alopecia refers to both the treatment of skin with a total absence of hair growth as well as the treatment of skin having reduced or patchy hair growth. Successful treatment results in an increased number of hairs.

Subjects to be treated according to the invention include human subjects as well as other mammalian subjects, such as dogs, cats, mice, rats, goats, llamas, minks, seals, beavers, ermines, and sheep. These can be treated for hair loss due or simply for enhancing wool or pelt production.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### EXAMPLE 1

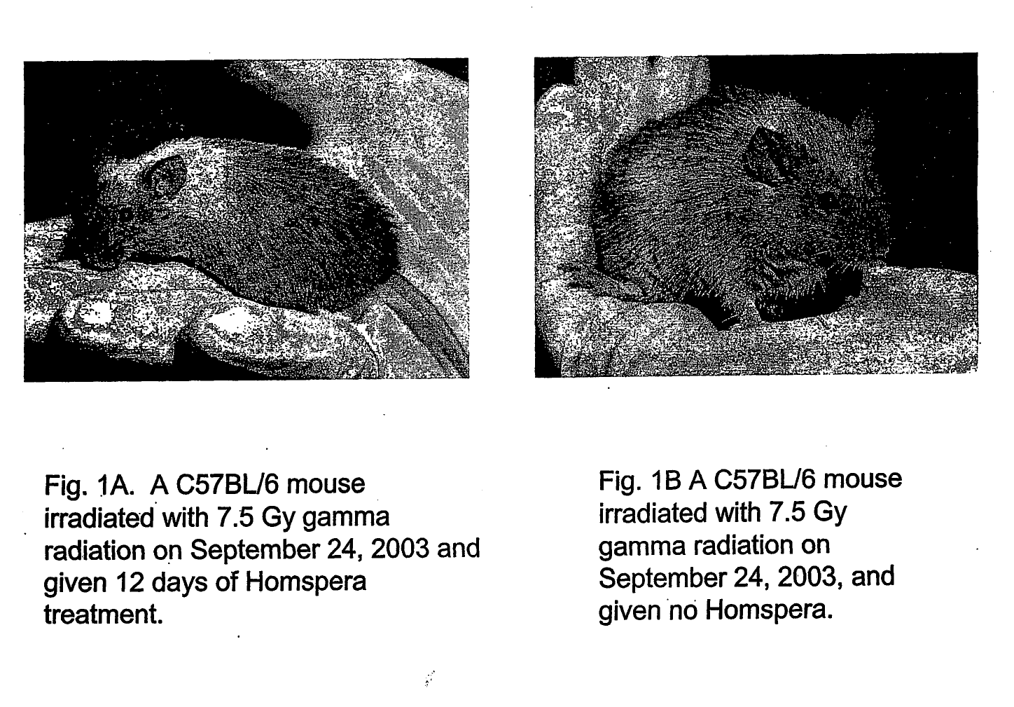

A C57BL/6 mouse was irradiated with 7.5 Gy gamma radiation on Sep. 24, 2003 and given 12 days of [Sar, Met(0₂)¹¹]-substance P (Homspera™) treatment. New growth (black) hair was observed at the rear of the mouse's back, See FIG. 1A. As a control, a C57BL/was 6 mouse irradiated with 7.5 Gy gamma radiation on Sep. 24, 2003 and given no Homspera™ treatment. No new growth hair was observed. See FIG. 1B.

### EXAMPLE 2

C57BL/6 mice were given a Cobalt 60 gamma radiation treatment on Sep. 24, 2002 of either 10 Gy, 7.5 Gy, 5 Gy, or 2.5 Gy. The 10 Gy mice died at an average of 11.3 days after the radiation exposure. All of the other mice in the other radiation exposure groups survived at least until Dec. 18, 2002. At 75 days after the initial radiation exposure, the 5 Gy and 2.5 Gy mice were administered a 10 Gy dose of Cobalt 60 gamma radiation. On the day of the second 10 Gy radiation exposure, it was noted that the 5 Gy and 2.5 Gy mice had hair loss over approximately 20% of their body with the most significant hair loss in their leg areas and around their ears. Half of the 5 Gy and 2.5 Gy (N=10) mice were administered a one micromolar Homspera™ dose by aerosol for 15 min/day beginning the day of the 10 Gy radiation exposure. The Homspera™ treated mice have restored their hair loss over the 9 days of Homspera™ treatment.

### EXAMPLE 3

C57BL/6 mice were divided into three groups. Groups 1 and 2 underwent a lethal dose of gammna radiation (7.75 Gy) on July 11th. Group 1 mice were treated with Homspera™ (50 micromolar for 35 days and then a maintenance dose of 10 micromolar until +90 days), and then they were killed at +90 days post-lethal radiation (October 9^{th}). Their immune system status was normal compared to control mice (Group 3) that had not been treated with radiation and had not been treated with Homspera™. Group 2 mice received Homspera™ treatment for 35 days after radiation, and then the Homspera™ treatment was completely stopped.

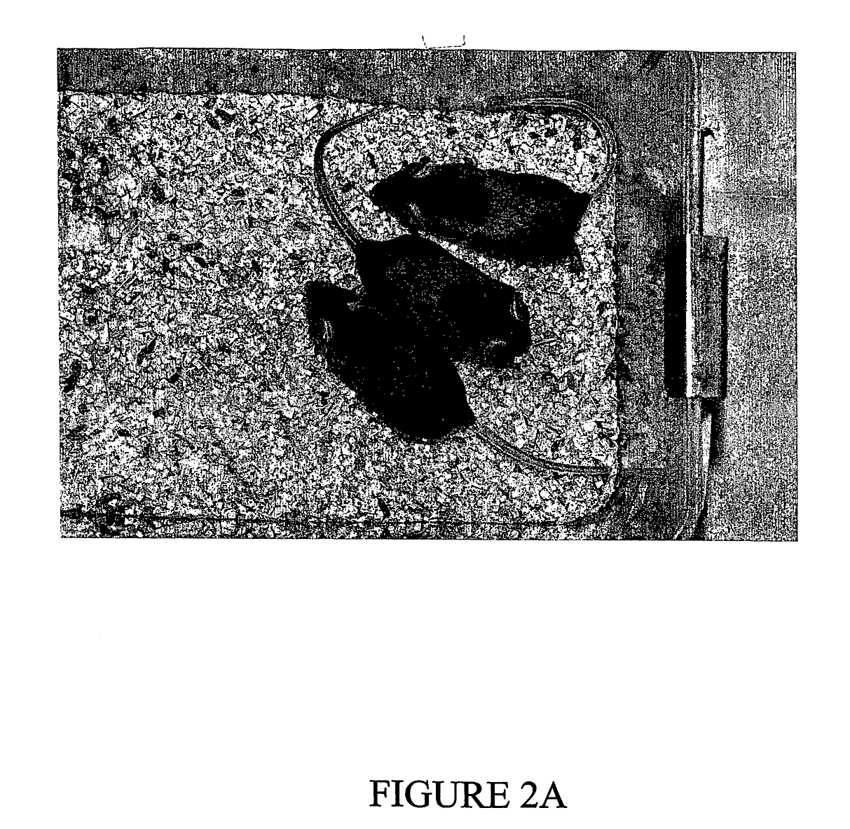

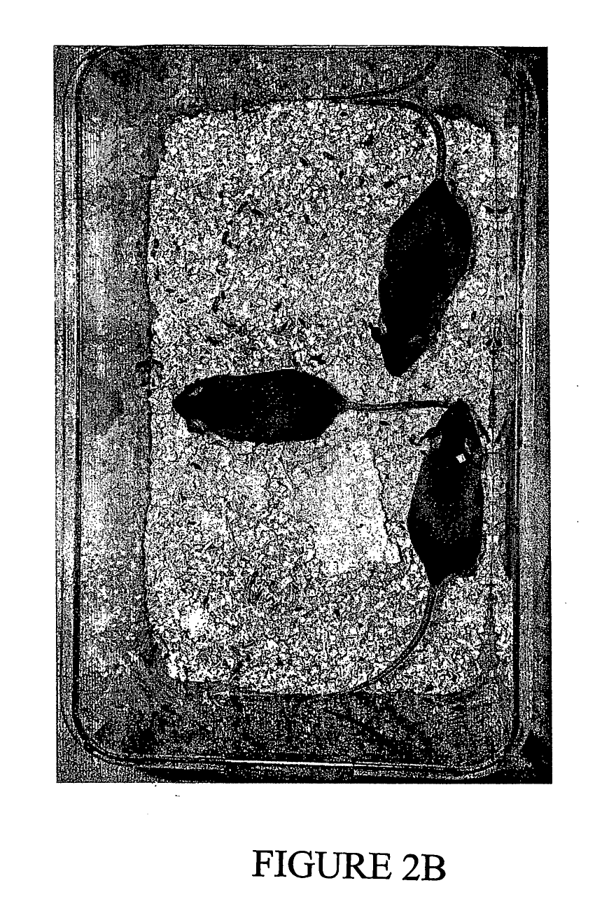

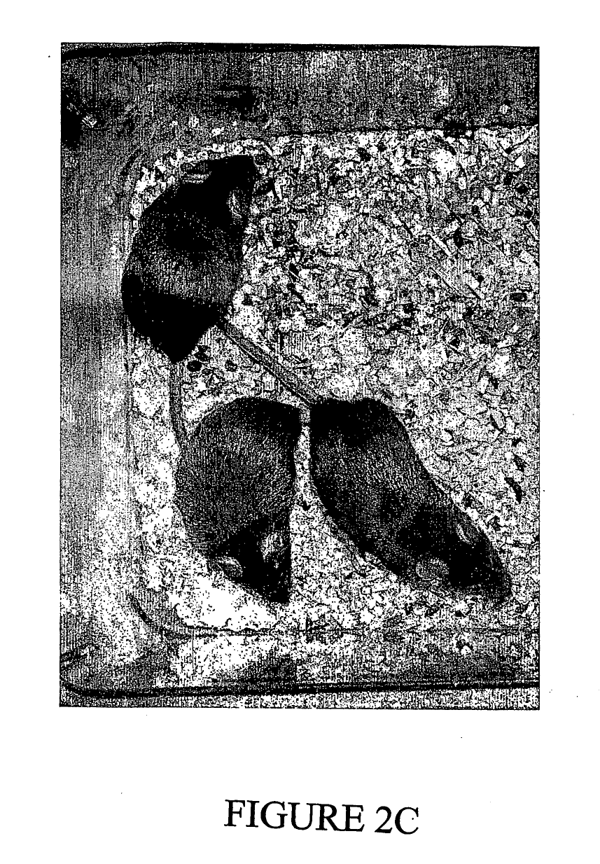

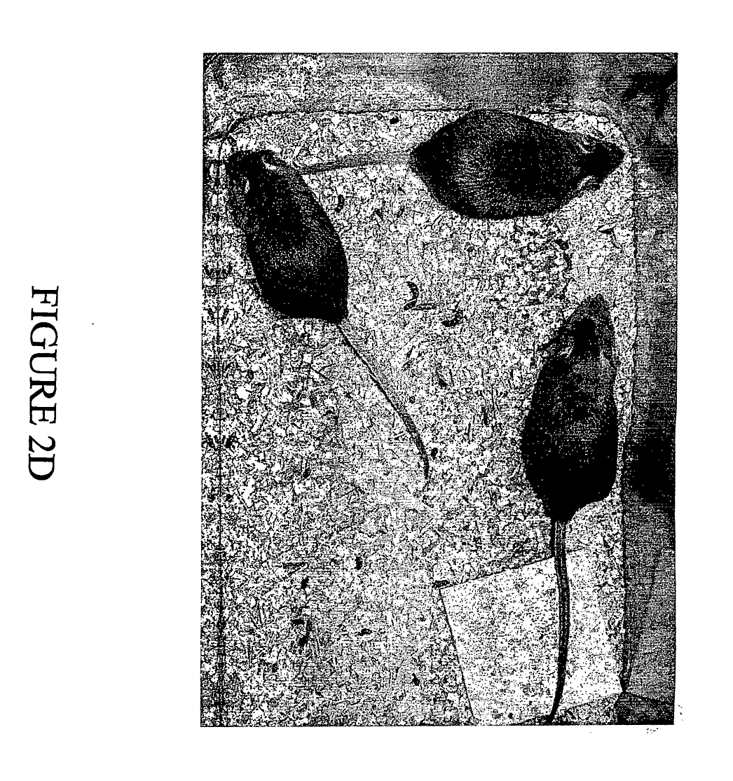

There are two sets of pictures of the Group 1 and Group 2 mice: one set of pictures taken on September 5^{th} at Day +56 after radiation (FIGS. 2A and 2B), and the other on October 9^{th} at Day +90 after radiation (FIGS. 2C and 2D). Both groups retained their hair.

1. A method of inducing new hair growth or retarding hair loss, comprising:
administering to a subject in need thereof substance P or a bioactive analog thereof selected from the group consisting of [Met-OH¹¹]-substance P, [Met-OMe¹¹]-substance P, [Nle¹¹]-substance P, [Pro⁹]-substance P, [Sar⁹]-substance P, [Tyr⁸]-substance P, [p-Cl-Phe^{7,8}]-substance P, [Sar⁹,Met(0₂)¹¹]-substance P, and analogs having the amino acid backbone RPKPQQFFGLM-NH₂, whereby new hair growth is induced or hair loss is retarded.

2. The method of claim 1 wherein the subject lost hair due to chemotherapy.

3. The method of claim 1 wherein the subject loss hair due to radiation exposure.

4. The method of claim 1 wherein the subject lost hair due to illness.

5. The method of claim 1 wherein a bioactive analogue is administered.

6. The method of claim 5 wherein the bioactive analogue is [Sar⁹,Met (0₂)¹¹]-substance P.

7. The method of claim 1 wherein the substance P analog or bioactive analog is administered as an aerosol.

8. The method of claim 7 wherein the concentration of substance P or the bioactive analogue in the administered aerosol is between 0.001 and 50 μM.

9. The method of claim 7 wherein the concentration of substance P or the bioactive analogue in the administered aerosol is between 0.05 and 10 μM.

10. The method of claim 7 wherein the concentration of substance P or the bioactive analogue in the administered aerosol is between about 0.1 and 5 μM.

11. The method of claim 1 wherein the substance P or bioactive analog is administered topically.

12. The method of claim 1 wherein the substance P or bioactive analog is administered subcutaneously.

13. The method of claim 1 wherein the substance P or bioactive analog is administered transdermally.

14. The method of claim 1 wherein Substance P is administered to the subject.

15. The method of claim 1 wherein a Substance P analog which has the amino acid backbone RPKPQQFFGLM-NH₂ is administered to the subject.
